(19) United States
(12) Patent Application Publication
(10) Pub. No.: US 2002/0116018 A1
(43) Pub. Date: Aug. 22, 2002

(54) Anastomosis system and methods for use

(76) Inventors: Walter J. Stevens, Woodbury, MN (US); Eunice Chen, Menlo Park, CA (US); Jessica Smith, Granite Bay, CA (US); Douglas Oguss, Northbrook, IL (US); Mary K. O'Connell, Palo Alto, CA (US); Jay Story, Boise, ID (US)

Correspondence Address: Bret E. Field, Bozicevic, Field and Francis LLP, Suite 200, 200 Middlefield Road, Menlo Park, CA, 94025, US

(21) Appl. No.: 10/010,960

(22) Filed: Nov. 9, 2001

Related U.S. Application Data
(63) Continuation-in-part of application No. PCT/US00/014689, filed on May 26, 2000.
(60) Provisional application No. 60/136,707, filed on May 28, 1999.

(51) Int. Cl. A61B 17/08

(52) U.S. Cl. 606/153

(57) ABSTRACT

An anastomosis system and methods for its use in end-to-side anastomoses are provided. The subject anastomosis system includes nesting, first and second structural means which are each tubular in structure and have a lip at at least one end. In performing an end-to-side anastomosis according to the subject invention, the subject anastomosis system is used to stably attach the graft vessel to the side of the host vessel in a manner that provides for fluid communication between the lumens of the graft and host vessels. Also provided are kits that include the subject systems. The subject anastomosis systems and methods find use in a variety of different anastomosis applications, including vascular anastomoses, and particular proximal anastomoses.

### CROSS REFERENCE TO RELATED APPLICATIONS

This application is a continuation-in-part of PCT/US00/14689 filed on May 26, 2000 which claims priority to the filing date of the U.S. Provisional Patent Application Serial No. 60/136,707 filed May 28, 1999, the disclosure of which is herein incorporated by reference.

### INTRODUCTION

1. Technical Field

The field of this invention is anastomosis, and more particularly end-to-side anastomosis.

2. Background of the Invention

Anastomosis is the union or connection of one tubular structure to another so that the interiors of the tubular structures communicate with one another. There are generally two types of anastomoses: end-to-end and end-to-side. In an end-to-end anastomosis, the ends of two different tubular structures are joined together. In an end-to-side anastomosis, however, the severed end of one tubular structure is connected around an opening cut into the side of a second tubular structure.

Anastomoses have been performed with a variety of different types of tubular structures or vessels in order to achieve a desired patient outcome. Typically, anastomoses are performed between airways, blood vessels, bowel segments, and urogenital tubes. The procedure for connecting blood vessels is referred to as vascular anastomosis.

One of the best known surgical procedures utilizing vascular anastomoses is the coronary bypass. In the context of coronary artery disease, the flow of oxygenated blood to the myocardium of the heart is impeded or compromised by a stenosis or obstruction in the coronary artery. This flow can be improved by providing a coronary artery bypass graft (“CABG”) which diverts blood flow around the stenosis, thereby restoring myocardial circulation. In these procedures, a graft (e.g. a saphenous vein graft, a LIMA graft or a synthetic graft) is harvested and attached to the aorta on one side of the stenosis utilizing an end-to-side proximal anastomosis and to a coronary artery on the other side of the stenosis utilizing an end-to-side distal anastomosis. In other words, a proximal end-to-side anastomosis and distal end-to-side anastomosis are performed, where the graft vessel is attached at the proximal site to the aorta and at the distal site to a coronary artery. In this specification, the terms proximal and distal are used according to their conventional meanings to those of skill in the art, where proximal refers to the end closest to the center/origin/source and distal refers to the end farthest away. As such, in terms of bypass procedures, proximal refers to the aorta or main/larger vessel while distal refers to the smaller, more distant graft vessel.

While a variety of different devices and methodologies have been developed over the years to perform anastomoses, including vascular anastomoses, there continues to be a need and an interest to develop new and better methods and devices for performing such procedures. Of particular interest would be the development of methods and devices that make end-to-side anastomoses simple and rapid to perform, thereby decreasing resource usage, e.g., operating room time, physician training, etc. With respect to vascular anastomosis devices, of particular interest would be the development of an anastomosis system that is capable of being used to perform a proximal end-to-side anastomosis, particularly while the heart is still beating and that establishes intima-to-intima contact of the two vessels being joined. Such improved methods and devices would lead to a number of advantages, including less expense, faster patient recovery, and the like.

### Relevant Literature

Patents of interest include: U.S. Pat. Nos. 2,453,056; 4,366,819; 4,368,736; 4,470,415; 4,523,592; 5,695,504; 5,702,412; 5,797,934; and 5,817,113.

### SUMMARY OF THE INVENTION

An anastomosis system and methods for its use in end-to-side anastomoses are provided. The subject anastomosis system includes nesting first and second structural means which are each tubular in structure and have a lip on at least one end. In performing an end-to-side anastomosis according to the subject invention, the subject anastomosis system is used to stably attach the graft vessel to the side of the host vessel in a manner that provides for fluid communication between the lumens of the graft and host vessels. Also provided are kits that include the subject systems. The subject anastomosis systems and methods find use in a variety of different anastomosis applications, including vascular anastomoses and particularly proximal anastomoses.

### BRIEF DESCRIPTION OF THE FIGURES

FIG. 1 provides a representation of a first structural means according to the subject invention.

FIG. 2 provides a representation of a second structural means according to the subject invention.

FIGS. 3A and 3B depict two different embodiments of a graft vessel attached to the first and second structural means of an anastomosis system according to the subject invention.

FIGS. 4A to 4D provide a step-by-step representation of the eversion of the distal end of a graft vessel over the distal surface of the lip of a first structural means in the process of producing a prepared graft vessel according to the subject invention.

FIG. 5 shows a prepared graft vessel according to the subject invention.

FIG. 6 provides a cross-sectional view of the site of attachment of the graft and host vessel following an end-to-side anastomosis according to the subject invention.

FIG. 7 provides a three-dimensional view of a rigid first structural means.

FIG. 8 provides a three-dimensional view of a rigid second structural means.

FIGS. 9 provides a three-dimensional view of the first and second structural means shown in FIGS. 7 and 8, respectively, in a partially nested configuration.

FIGS. 10A to 10C provide different views of a delivery device according to the subject invention.

FIGS. 11A to 11N provide a sequential showing of the various steps of securing a graft vessel to the side of a host vessel using the methods and devices of the subject invention.

### DESCRIPTION OF THE SPECIFIC EMBODIMENTS

Anastomosis systems and methods for their use in performing end-to-side anastomoses are provided. The subject systems include nesting first and second structural means that have a tubular structure and a lip on at least one end. In practicing the subject methods, the anastomosis system is used to stably attach the graft to the host vessel in a manner such that fluid communication is established between the graft and host vessels. Also provided are kits for use in performing end-to-side anastomoses according to the subject invention. In further describing the subject invention, the anastomosis system and components thereof (e.g., first and second structural means) are described first, both generally and in terms of specific embodiments depicted in figures, followed by a discussion of the subject methods of performing end-to-side anastomoses and kits that include the subject anastomosis systems.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Anastomosis System

As summarized above, the subject invention provides an anastomosis system that enables one to stably attach the end of a graft vessel to the side of a host vessel in a manner such that fluid communication between the lumens of the graft and host vessels is established. The systems of the subject invention are made up of nesting first and second structural means. The first and second structural means share many common features. The first and second structural means have a tubular structure with a lip at one end. The lip of each structural means in certain embodiments is characterized by expanding radially outward in substantially all, if not all, directions from the base of the tubular region of the structural means. The shape of the lip may have any convenient shape configuration, including square, rectangular and curvilinear shape, such as oval, circular, irregular etc., where in many embodiments, the lip has a curvilinear configuration, i.e., the perimeter of the lip is curvlinear in shape.

A feature of the first and second structural means of the subject systems is that the first and second structural means are nesting, by which is meant that the tubular region of one of the structural means (typically the first structural means) fits inside the tubular region of the other structural means (typically the second structural means). In other words, the two structural means can be fit together in a manner such that the tubular member of one of the structural means can be positioned inside the tubular member of the other structural means. As such, the first and second structural means of the subject systems are capable of assuming a nested configuration. As the first and second structural means are capable of assuming a nested configuration, the outer diameter of one of the structural means is necessarily shorter than the inner diameter of the other structural means, e.g., the outer diameter of the first structural means is shorter than the inner diameter of the second structural means. The difference in diameters should be large enough to provide for insertion of one of the tubular members into the other tubular member but small enough to provide for a substantially snug fit when positioned in an end-to-side anastomosis.

The structural means may be made up of rigid materials, flexible materials, or be a composite of both rigid and flexible materials. Whether the materials are flexible or rigid, they should be biocompatible in view of the use of the subject structural means. By biocompatible is meant that they should be capable of being implanted and maintained in an animal host for a substantial period of time with little or no, and preferably no, toxic effects for the animal host. Examples of suitable rigid biocompatible materials include, but are not limited to: medical grade alloys, such as cobalt-chromium alloy, titanium alloy, stainless steel, ceramics and composite materials, and the like. Examples of flexible materials include elastic materials, where suitable elastic materials are materials that exhibit elasticity at a relevant temperature range, i.e., below room temperature to body temperature, e.g., from about 10 to 50 ° C. at least, and are biocompatible. One type of biocompatible elastic material of interest is the class of memory alloys, including those described in: U.S. Pat. Nos. 5,876,434; 5,797,920; 5,782,896; 5,763,979; 5,562,641; 5,459,544; 5,415,660; 5,092,781; 4,984,581; the disclosures of which are herein incorporated by reference, e.g., biocompatible alloys that find use include those nickle-titanium (NiTi) shape memory alloys sold under the Nitinol™.

Whether or not the first and second structural means are fabricated from rigid or flexible materials, the first and/or second structural means may be modified in a number of different ways so as to alter the properties of the materials from which they are fabricated, e.g., the surface properties of one or more of the elements. As such, in many embodiments, one or more components of the structural means may be coated with a biocompatible polymeric membrane coating. The membrane coating may be fabricated from any convenient biocompatible polymer, where suitable biocompatible polymers include, but are not necessarily limited to: biocompatible polymers and/or elastomers. Suitable biocompatible polymers include, but are not necessarily limited to, materials such as, for example, polyethylene, homopolymers and copolymers of vinyl acetate such as ethylene vinyl acetate copolymer, polyvinylchlorides, homopolymers and copolymers of acrylates such as polymethylmethacrylate, polyethylmethacrylate, polymethacrylate, ethylene glycol dimethacrylate, ethylene dimethacrylate and hydroxymethyl methacrylate, polyurethanes, polyvinylpyrrolidone, 2-pyrrolidone, polyacrylonitrile butadiene, polycarbonates, polyamides, fluoropolymers such as polytetrafluoroethylene and polyvinyl fluoride, polystyrenes, homopolymers and copolymers of styrene acrylonitrile, cellulose acetate, homopolymers and copolymers of acrylonitrile butadiene styrene, polyvinylchloride, silicone rubber, polymethylpentene, polysulfones, polyesters, polyimides, polyisobutylene, polymethylstyrene and other similar compounds known to those skilled in the art. Suitable, biocompatible elastomers include, but are not necessarily limited to, biocompatible elastomers such as medical grade silicone rubbers, polyvinyl chloride elastomers, polyolefin homopolymeric and copolymeric elastomers, urethane-based elastomers, and natural rubber or other synthetic rubbers, fluorinated polymers (e.g., PTFE), and the like. It should be understood that these possible biocompatible materials are included above for exemplary purposes and should not be construed as limiting.

In certain embodiments, the biocompatible polymer may have one or more active agents or drug compounds associated with it, such that the structural means also serve as a drug delivery means, e.g., a slow release drug delivery means. A variety of different active agents may be delivered using the structural means of the subject invention, where such active agents include small organic molecules, either synthetic or naturally occurring; peptides; proteins; nucleic acids; etc. Active agents of interest include: agents having anti-restinosis activity, such as those disclosed in U.S. Pat. Nos. 5,492,926; 5,462,937; 5,457,113; 5,599,227; 5,569,647; 5,428,151; active agents having anti-cell proliferation activity, active agents having anti-thrombotic activity; as well as other agents that may be desirable; and the like.

Despite the above common features of the first and second structural means of the subject invention, differences between the first and second structural means do exist. Accordingly, each of the first and second structural means is now described in greater detail separately below.

First Structural Means

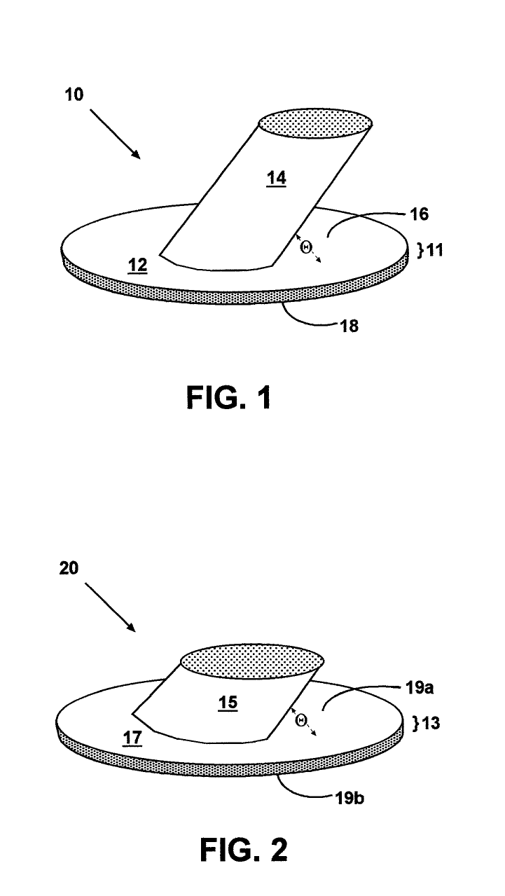

As described above, the first structural means is a tubular structure that is flared at one end to form a “lip” feature extending beyond the outer diameter of the tubular region. FIG. 1 provides a three-dimensional view of a first structural means according to the subject invention, where the first structural means is a simple configuration made up of an elongated tubular member that is flared at one end to form a lip. As can be seen in FIG. 1, the first structural means 10 has an elongated tubular region or member 14 with a flared end or lip 12 at one terminus. Lip 12 has an upper surface 16 and a lower surface 18.

The lip 12 has an area that is sufficiently large to provide for stable attachment of the graft vessel to the side of the host vessel following an anastomosis procedure, as described in greater detail below. In other words, the lip has an area that is sufficiently large such that it provides for stable attachment of the graft to the host vessel and cannot be readily pulled out of the opening in the side of the host vessel into which it has been inserted in the subject methods. The area of the lip (defined by the outer perimeter of the lip and including the area of the empty space or opening located where the tubular end expands into the lip) may vary considerably depending on the nature of the two vessels to be joined. For example, where the anastomosis system is designed to be used in a vascular end-to-side anastomosis, particularly a proximal end-to-side anastomosis of a graft vessel to the aorta, the area of the lip is at least about 5 mm², usually at least about 10 mm² and more usually at least about 20 to 30 mm² and in some embodiments at least about 35 mm². The area of the lip typically does not exceed about 60 mm² and usually does not exceed about 40 mm².

As mentioned above, the shape of the flared end or lip of the first structural means may be any convenient shape, e.g., square, rectangular, triangular, trapezoid, circular, oval etc., but is generally a curvilinear shape, where any convenient curvilinear shape may be employed, such as: circular, oval, or other convenient curvilinear shape, including an irregular curvilinear shape, a curvilinear shape with scalloped edges, etc. The lip should be as thin as possible such that fluid flow in the host vessel following anastomosis is not substantially impeded or compromised by the presence of the lip and graft tissue everted over it, as described in greater detail supra. As such, the thickness 11 typically ranges from about 0.005 to 1.0 mm, usually from about 0.01 to 0.5 mm and more usually from about 0.05 to 0.25 mm.

The length of the tubular member or element of the first structural means may vary greatly depending on the nature of the vessels to be joined, where the length is typically just long enough to provide the required support function at the site at which the graft vessel is attached to the host vessel. The tubular region of the structural means has a generally curvilinear, and usually substantially circular, cross-sectional shape extending its entire length. The inner diameter varies in length depending on the particular graft vessel for which the first structural means is designed to be used. For example, in vascular anastomoses, particularly proximal anastomoses in which a graft vessel as anastomosed to the side of the aorta, the inner diameter is generally at least about 2 mm, usually at least about 3 mm and more usually at least about 4 mm. The outer diameter of the structural means is chosen so as to fit inside the second structural means, described in greater detail infra, such that the tubular region of the first structural means is capable of nesting inside the tubular region of the second structural means, as described above. In many embodiments, the length of the first tubular means ranges from about 1 to 25, usually from about 2 to 20 and more usually from about 2 to 15 mm.

In certain embodiments, the joint angle θ between the outer wall of the tubular region and the lip (See FIG. 1) is chosen so as to facilitate fluid flow from the host vessel into the graft vessel. Generally the joint angel θ is between about 30 and 75°, usually between about 40 and 60°, where in many embodiments the joint angle θ is about or is between about 45 and 60°.

The first structural means may optionally comprise one or more locking means which prevents removal of the second structural means from the first structural means following attachment of the graft and host vessels, as described in greater detail infra.

This locking means may take on a variety of different configurations, so long as it prevent separation of the first and second structural means following their placement into a nesting position during use, e.g., so long as it prevents unwanted backwards movement (i.e., movement away from the host vessel) of the second structural means relative to the first structural means. This locking means may be permanent or reversible, i.e., the locking means may be one-way or two-way. An example of a one-way locking means may be one or more spaced apart protrusions on the outer surface of the tubular region of the first structural means which act like “barbs” to provide for one-way movement of the second structural means onto the first structural means. Another example of a locking means may be ratchet means, e.g., as made up by tooth on one of the structural means and corresponding groove on the other of the structural means, that provides for the one-way movement, as described in greater detail below. Examples of two-way or reversible locking means include ratchet elements that can be locked and released, e.g., by turning one element relative to another, as describe in greater detail below. As such, the locking means may be a single element present on either the first or second structural means, or a combination of elements present on both the first and second structural means.

In certain embodiments, the first structural means may also include a mechanical engaging means for securing the first structural means to tissue, e.g., for securing the first structural means to the graft vessel. This engaging means may be in any convenient form, such as one or more, usually a plurality of, spikes extending upward from the proximal surface of the lip of the first structural means. The spikes may vary in length, where in certain embodiments they will be just long enough to secure the lip to the host vessel, but not protrude through the host vessel wall. The spikes may or may not include barbed ends.

Second Structural Means

The second structural means of the subject anastomosis systems is analogous to the first structural means. As with the first structural means, the second structural means 20 as shown in FIG. 2 has a tubular shape 15 which flares or expands into a lip feature 17 at one end, where the lip feature 17 has an upper surface 19a, a lower surface 19b and a thickness 13. The second structural means may also have a second lip on the distal end, allowing a means of pressing the second structural means onto the first structural means during use. As the first and second structural means have a “nesting” relationship, the second structural means is designed to fit over the tubular region of the first structural means. As such, a critical feature of the second structural means is that the inner diameter of the tubular region is greater than the outer diameter of the tubular region of the first structural means. The magnitude of this difference in lengths is often merely sufficient to provide sufficient space for the second structural means to fit (with application of external force) over the tubular end of the first structural means but not so great that the second structural means can readily slide off of the tubular region of the first structural means when tissue is “sandwiched” between the two structural means, as described in greater detail supra. The length of the inner diameter of the tubular member of the second structural means typically ranges from about 2 to 15, usually from about 4 to 10 and more usually from about 5 to 7 mm. The length of the outer diameter of the tubular member of the second structural means typically ranges from about 2 to 15, usually from about 4 to 7 and more usually from about 5 to 10 mm.

Generally, the length of the tubular region must be sufficient to provide for the requisite support and stable attachment of the graft to the host vessel. In many embodiments, the length of the second tubular means ranges from about 1 to 10, usually from about 2 to 7 and more usually from about 3 to 6 mm.

The configuration of the lip or flared end of the second structural means may or may not be the same as that of the first structural but, as with the first structural means, may be any convenient shape, including curvilinear shape, e.g., circular or oval, as mentioned above. The size of the lip of the second structural means may be the same or different than the size of the lip of the first structural means. In certain embodiments, the area of the second lip typically ranges from about 5 to 100, usually from about 25 to 100 and more usually from about 30 to 60 mm.

The second structural means may optionally have a locking means for mechanically securing the second structural means to the first structural means, e.g., a component of a two component ratchet system. In addition, the second structural means may have one or more engaging features on the lip for securing the lip to tissue, e.g., spikes, etc.

Fabrication of the First and Second Structural Means

The structural means may be fabricated using any convenient protocol, where the particular protocol will vary depending on the nature of the specific materials from which the structural means is fabricated. Representative protocols that may be employed to produce the subject structural means of the anastomosis system include: machining, molding, forming, stamping, laser cutting, laser welding and the like.

Anastomosis Methods

The above described first and second structural means find use in the end-to-side anastomosis of a graft vessel to a host or primary vessel, e.g., as is done in the proximal anastomosis portion of a coronary artery bypass procedure. In the anastomosis methods of the subject invention, the following steps are performed: (a) graft vessel preparation; and (b) attachment of the prepared graft vessel to the side of the host vessel in a manner such that fluid (including gaseous) communication between the lumens of the host and graft vessels is established. Each of these steps is described in greater detail separately below.

Graft Vessel Preparation

To prepare a graft vessel for attachment to the side of a host vessel according to the subject methods, the first step is to harvest the vessel. Depending on the nature of the vessel, e.g., artery, vein, colon, renal tubule, bronchial tubule, etc., as described in greater detail below, the harvesting protocol employed may vary greatly. Convenient methods for harvesting such vessels are known to those of skill in the art. Because of the nature of the subject methods and devices employed therein, however, it is critical that the outer surface of the graft vessel be “clean,” by which is meant that excess tissue and the like is removed from the graft vessel prior to use. Any convenient procedure may be employed to remove excess tissue from the vessel and expose the adventitia of the vessel, so long as the procedure does not substantially disrupt the integrity of the graft vessel such that the graft vessel is no longer suitable for carrying fluid.

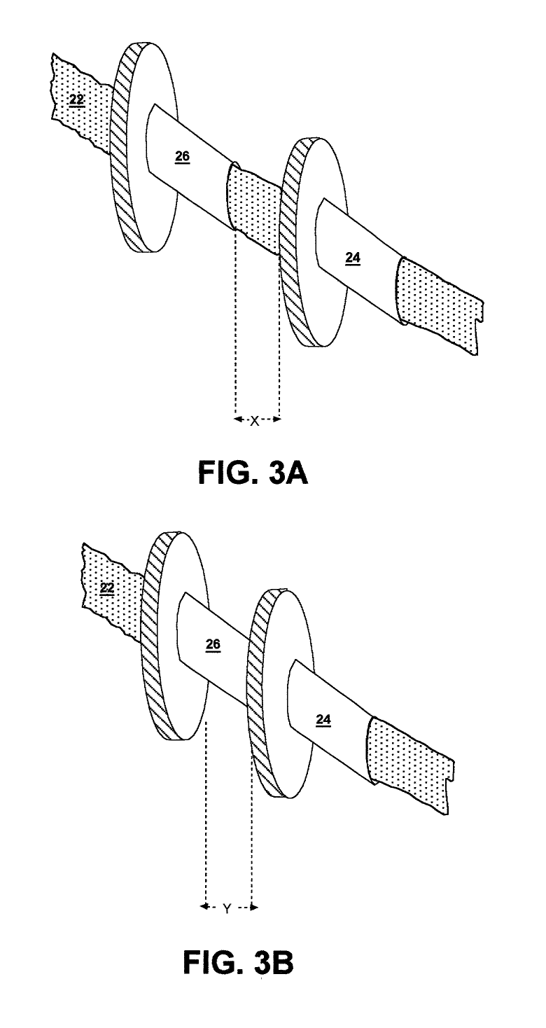

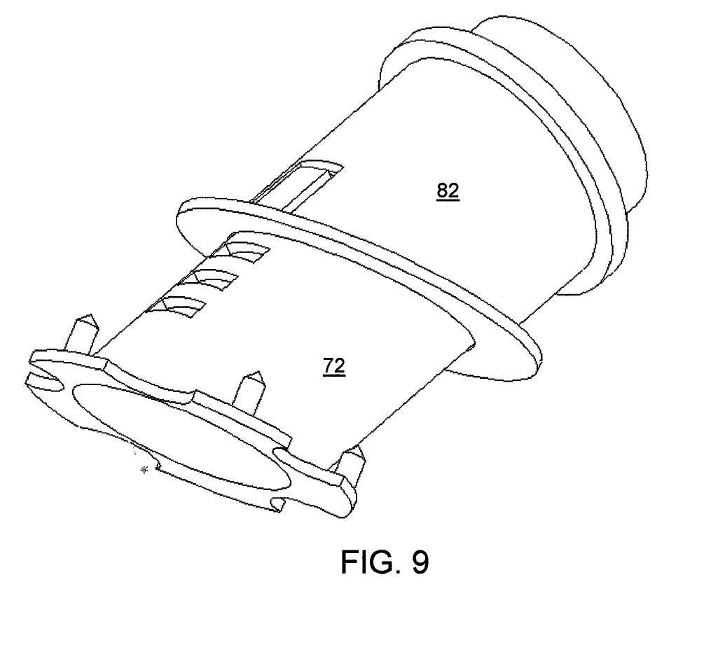

Once a suitable vessel is harvested and cleaned, the graft vessel is sequentially passed through the first and second structural means (described supra) such that the first structural means is closer to the terminus of the graft vessel than the second structural means, i.e., so that the first structural means is adjacent to the graft vessel terminus. During graft vessel preparation, the first and second structural means may or may not be connected to each other, i.e., partially nested. Thus, in certain embodiments, the graft vessel may be threaded through the second structural means first and the first structural means last, such that the second structural means is a variable distance from the first structural means. FIG. 3A provides a representation of this embodiment, where graft 22 is threaded through second structural means 24 and first structural means 26, where the first and second structural means are separated by distance X. In a second embodiment, the two structural means may be partially nested during graft vessel preparation, such that the vessel is threaded through both the structural means in one combined step. By partially nested is meant that the second structurally means is slid partially over the first structural means. See FIG. 3B. See also FIG. 9. Where the structural means are partially nested, the distal surface of the lip of the second structural means is separated from the proximal surface of the lip of the first structural means (distance Y as indicated in FIG. 3B) by a sufficient distance such that the lip of the first structural means can be inserted into the host vessel without concomitant insertion of the lip of the second structural means.

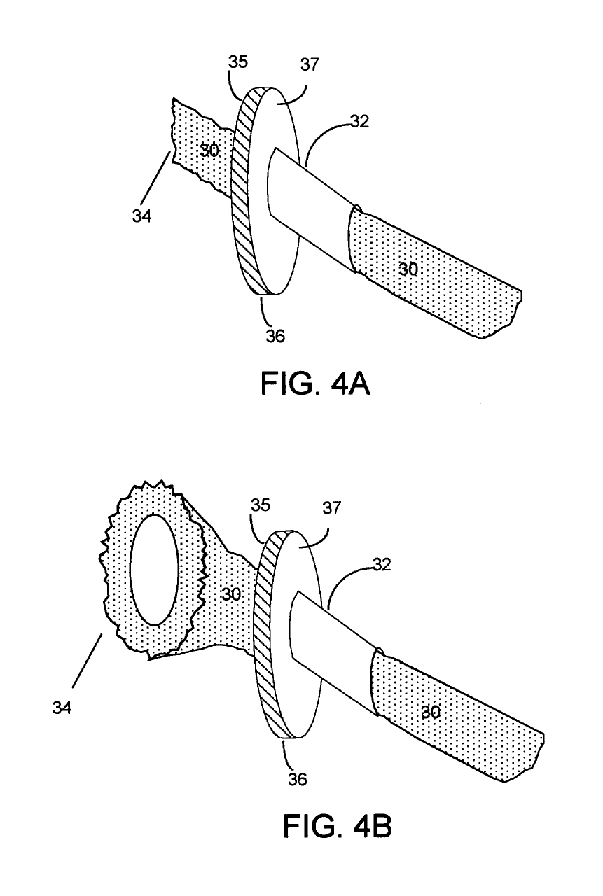

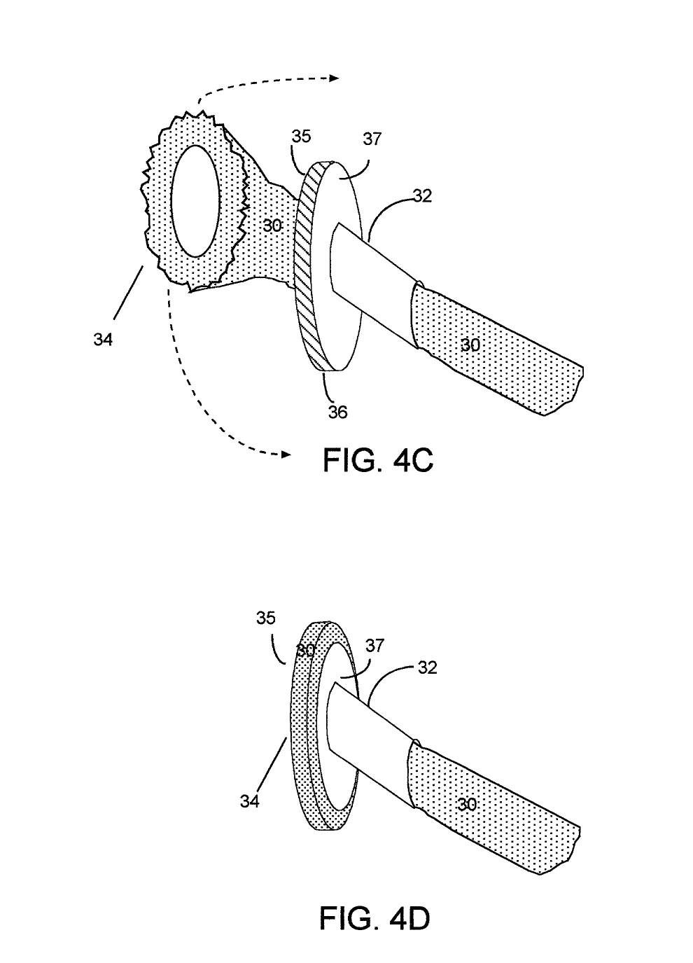

The next step in the graft vessel preparation is to evert the proximal end of the graft vessel over the lip of the first structural means such that the intimal side of the graft vessel covers the entire bottom or lower surface of the lip of the first structural means and at least a portion of the upper surface of the lip, including up to the entire upper surface of the lip. This step of the subject methods is shown in FIGS. 4A to 4D. FIG. 4A shows graft vessel 30 having a first structural means 32 positioned near its end 34. First structural means 32 has lip 36 with lower surface 35 and upper surface 37. FIG. 4B shows the end of the graft vessel 34 being everted or folded in a manner sufficient to expose the intimal surface of the graft vessel. FIG. 4C depicts the movement of the everted end 34 onto the lip of the first structural means, such that the exposed intimal surface of the graft vessel covers the entire lower surface 35 of the lip 36 of the first structural means and at least a portion of the upper surface 37 of lip 36, as shown in FIG. 4D. Generally, the everted end of the graft vessel is brought together with lower surface of the lip of the first structural means by: (a) pulling the graft vessel away from the first structural means in the direction of the arrows shown in FIG. 4C; (b) pushing the first structural means onto the everted end of the graft vessel; or (c) a combination of the above two movements. The everted end of the graft vessel is further folded over the lip in the direction of the arrows shown in FIG. 4D to at least partially cover the upper surface of the lip.

In many embodiments, the everted end of the graft vessel is typically secured onto the lip of the first structural means using a securing means. The everted end of the graft vessel may be secured to the lip using any convenient means including suturing, stapling, radiofrequency, heat, lasers, adhesives, etc., e.g., spikes, as discussed in greater detail below.

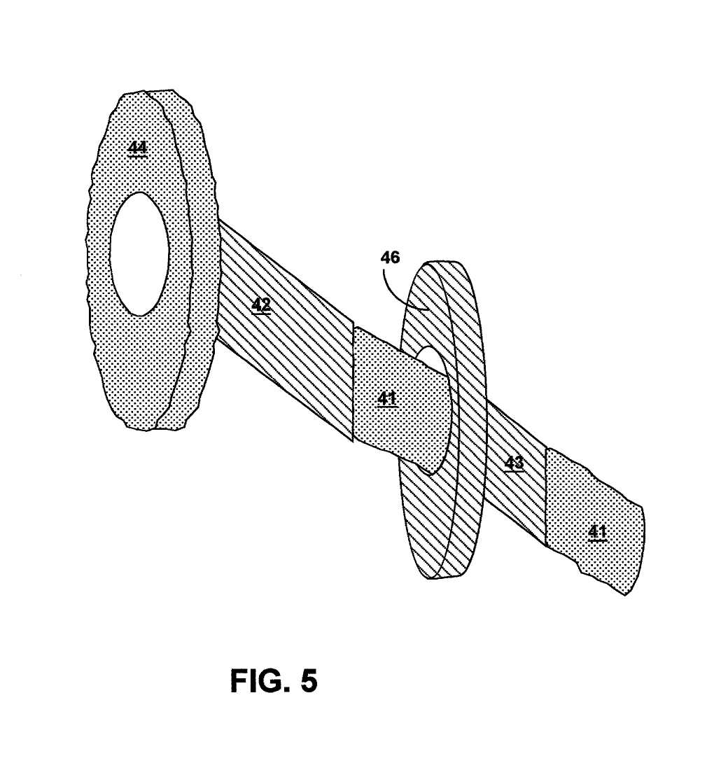

A representative prepared graft vessel according the subject invention is shown in FIG. 5. In FIG. 5, prepared graft 40 comprises graft vessel 41 threaded through the first and second structural means or devices, 42 and 43, of the anastomosis system of the subject invention. The end of the graft vessel has been everted over the lip of the first structural means, such that the entire bottom surface and at least a portion of the upper surface of the lip of the first structural means is covered by the everted end of the graft vessel.

Attachment of Prepared Graft Vessel to Primary Vessel

The next step in the subject methods is to stably attach the prepared graft vessel described above to the side of a primary or host vessel in a manner such that fluid (including gas) in the host vessel is capable of flowing through the attached graft vessel. Specifically, when the graft vessel is attached to the host vessel according to the subject invention, a structure is produced that is characterized by having intima-to-intima contact between the graft and host vessels and host tissue sandwiched between the lips of the first and second structural means, where in many embodiments the structure is further characterized in that intimal tissue from the graft and host do not touch the first and second structural means, i.e., only advential tissue of the graft and host contact or touch the first and second structural means.

To attach the prepared graft vessel to the side of the primary or host vessel, the first step is to form an opening in the side of the host vessel. The opening must be sufficiently large to allow the desired amount of fluid, e.g. blood, flow from the host vessel into the graft vessel and insertion of the lip into the lumen of the host vessel. The opening may be a straight line incision, circular cutaway, etc. The opening may be prepared using any convenient methodology, such as manual incision, remote incision with minimally invasive tools or punch tool, e.g., catheters, etc.

The next step is to insert the end of the prepared graft vessel (i.e., the graft vessel in combination with the first and second structural means with the end of the graft vessel everted over the lip of the first structural means) through the opening in the side of the host vessel.

The graft vessel is then moved away from the host vessel until resistance is felt, which indicates that the lip of the first structural means is flush with the intimal surface of the host vessel. The lip of the first structural means does not readily come out of the opening in the side of the host vessel since the cross-sectional area of the lips exceeds that of the opening in the side of the host vessel.

The next step is to attach the graft vessel to the side of the host vessel in a manner such that the graft vessel is stably secured to the side of the host vessel and fluid communication is established between the lumens of the graft and host vessels. Attachment of the graft to the host vessels is accomplished by moving the second structural means over the tubular region of the first structural means until the second structural means is positioned on the outer surface of the host vessel, i.e., the bottom surface of the lip of the second structural means is adjacent to or lying on the outer surface of the host vessel. In certain embodiments, the second structural means fits sufficiently tight around the tubular region of the first structural means such that it does not readily move from its position once placed next to the host vessel wall. However, the second structural means may be further secured in position through use of a locking means, sutures, staples, radiofrequency sealing, heat sealing, laser sealing, adhesives, etc. In addition, one or more spikes or analogous structures may provide for screening of the bottom surface of the lip to the host vessel.

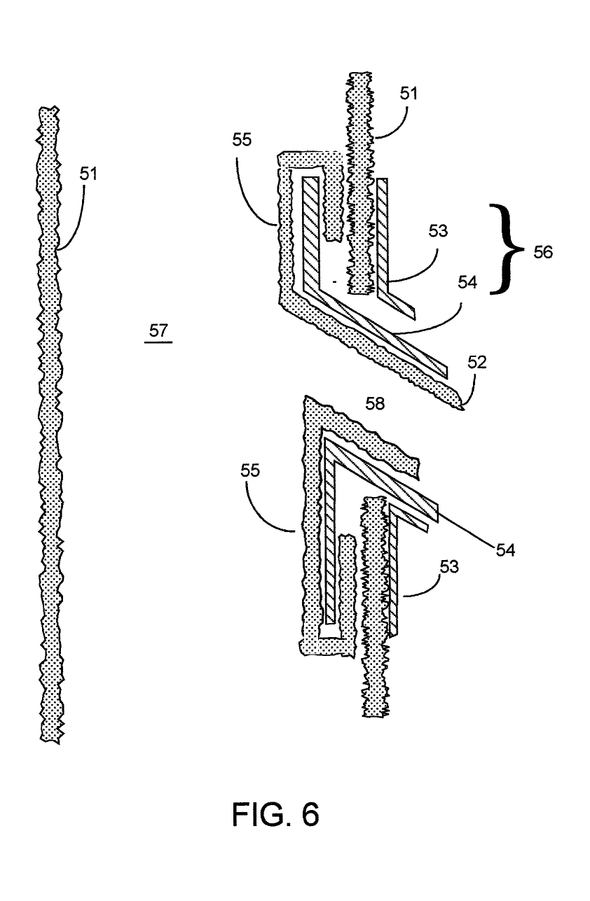

FIG. 6 provides a cross-sectional view of the attachment of a graft vessel to a host vessel according to the subject invention. Graft vessel 52 is attached to side of the host vessel 51 with structural means 53 and 54. The end of graft vessel 55 is everted over the bottom surface and at least a portion of the upper surface of the lip of structural means 54 in a manner sufficient to limit the contact of the structural means to advential surfaces of the graft and host vessels. Structural means 53 is positioned over the outer surface of structural means 54 in a manner such that host vessel 51 is sandwiched between the two structural means. As seen in FIG. 6, the lumens of the host and graft vessel, 57 and 58, are in fluid communication. This structure is maintained and stabilized through pressure of the second structural means on to the first structural means, where the pressure results from one or more features, such as snug fit, locking means, spikes for securing to tissue, etc.

The above steps may be performed using any convenient protocols and devices, where the procedure may be an open field, minimally invasive or marginally invasive procedure.

Representative Specific Embodiments

As mentioned above, the subject anastomosis systems may be made up of rigid structural means, flexible structural means or composite flexible/rigid structural means. This next section provides: (a) a description of a first representative embodiment of the invention in which the structural means are rigid; and (b) a description of a second representative embodiment of the invention in which at least the lips of the structural means are flexible.

Anastomosis System Made Up of Rigid Structural Means

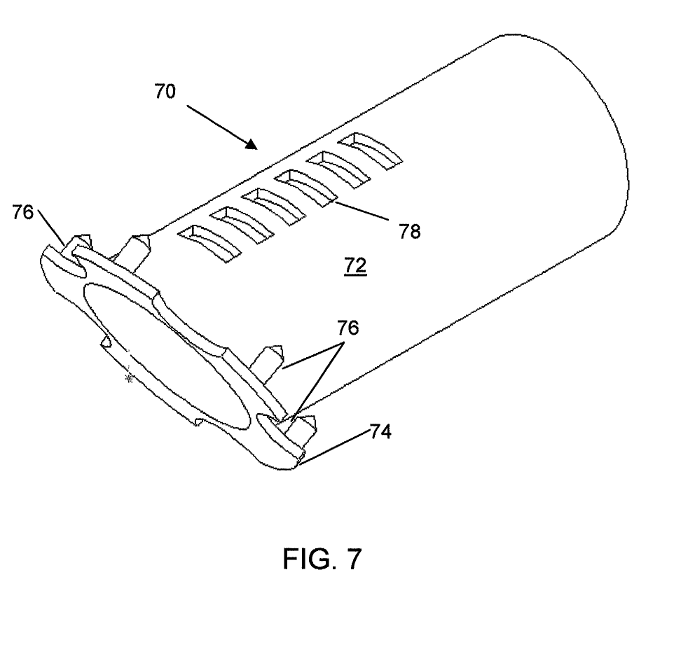

FIGS. 7 to 9 provide representations of an anastomosis system according to the subject invention that is made up of rigid first and second structural means or fixtures. As indicated above, the rigid structural means may be fabricated from any convenient material, e.g., stainless steel, medical grade alloys, such as titanium alloy, cobalt chromium alloy, etc.

FIG. 7 provides a three dimensional overhead view of a rigid first structural means according to this embodiment of the subject invention. In FIG. 7, structural means 70 is made up of elongate tubular member or region 72 and lip 74. Lip 74 has a cross sectional configuration that is characterized by curvilinear, particularly scalloped, perimeter. Lip element 74 further includes a plurality of spikes 76. Shown on the outer surface of tubular member 72 are grooves or slots 78 which work in conjunction a corresponding tooth or analogous protrusion on the second structural means to make a racthet locking means, where these grooves and teeth can be configured to provide for either a one-way irreversible or a two-way, reversible, ratchet locking means.

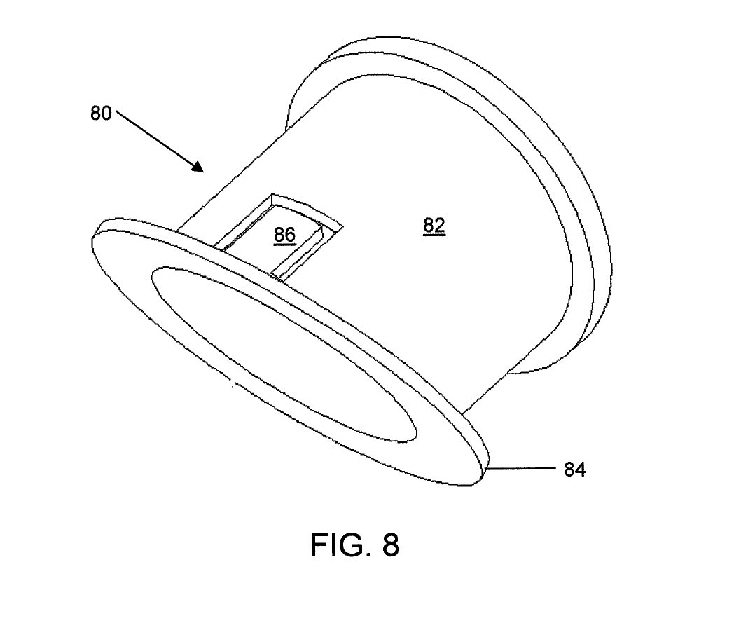

FIG. 8 provides a three dimensional view of a rigid second structural means 80. Structural means 80 is made up of elongate tubular member 82 and lip 84. Present on tubular member 82 is the corresponding ratchet locking member 86 which bends down into the groove members 78 of the first structural means shown in FIG. 7 when the two are in nesting configuration in order to provide for a one-way locking of the position of the second structural means relative to the first. To provide for a two-way or reversible locking means, the tooth element can be made with beveled corners, i.e., corners cut at a slight angle, such that the structural means can be twisted relative to each other, which twisting movement removes the tooth from the groove and allows the second structural means to be moved away from or separated from the first structural means. It is emphasized that this beveled tooth/slot ratchet means is only one example of a two-way locking means, and other locking means fall within the scope of the invention.

FIG. 9 provides a three-dimensional overview of the first and second structural means in a partially nested configuration, showing how the two structural means fit together.

In using this anastomosis system to perform an end-to-side anastomosis, a graft vessel is prepared as described above, such that the graft vessel is threaded sequentially through the second and first rigid structural means and the end of the graft vessel is everted over the lip of the first structural means in a manner such that the entire bottom surface and at least a portion of the upper surface are covered by the everted graft end. The graft covered lip of the first structural means is then inserted into a slit or analogous opening in the host vessel, e.g., aorta, and pulled back until resistance is felt, where the host vessel may be prepared as appropriate, e.g., by restricting blood flow at the site of anastomosis, e.g., with a clamp; etc. Next, the first structural means is moved towards the lip of the second structural means to produce a nested configuration that is sufficient to secure the graft vessel to the host vessel and establish fluid communication between the lumens of the vessels.

Anastomosis System Made Up of Flexible Structural Means

In a representative embodiment of an anastomosis system where the first and second structural means are flexible, the first and second structural means are configured as shown in FIGS. 1 and 2. In this embodiment, at least the lip feature of the first and second structural means is fabricated from a flexible material that can be folded so as to be capable of being inserted into an opening that is smaller than the unfolded lip structure. Note that other components of the structural means in this embodiment may be rigid or flexible. This step generally involves folding or manipulating the lip of the first structural means (having the everted end of the graft vessel stably attached to it) in a manner such that it can pass through the opening in the side of the host vessel. Once the end of the graft vessel and lip of the first structural means is present inside the lumen of the host vessel, the lip of the first structural means is allowed to assume its unconstrained or resting configuration.

In many embodiments, both the first and second structural means that make up the subject anastomosis systems are elastic, by which is meant that at least a portion of the structural means (e.g., the lip) is flexible and may be manipulated in various ways and, following removal of any externally applied force (other than ambient forces, e.g., gravity) assumes its substantially initial shape. Thus, certain features of the subject structural means (e.g. the lips) may be folded during a certain part of their use and then resume their initial unfolded shape following completion of the procedure in which they are used, as described in greater detail infra. In using these flexible structural means, a graft vessel is first prepared as described above. In certain embodiments, an adhesive or glue is employed to secure the everted end of the graft to the lip of the first structural means. Where an adhesive or glue is employed, the method further comprises applying the adhesive composition to at least one of the outer or adventitial surfaces of the everted end of the graft vessel and the distal surface of the lip of the first structural means.

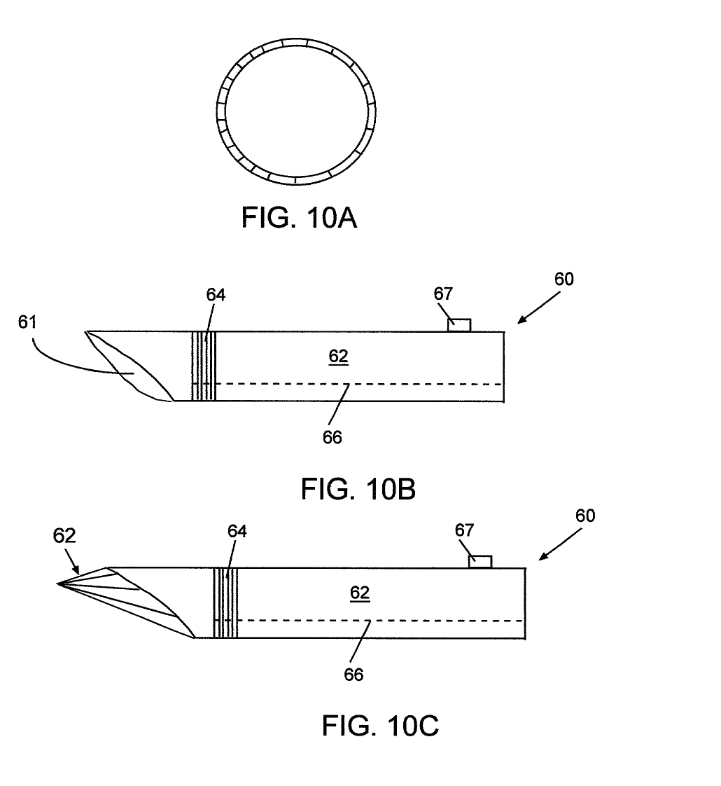

Following preparation of the graft vessel, the graft vessel is attached to the host vessel generally as described above. One convenient means of stably attaching the prepared graft to the side of a host vessel is to employ a delivery means such as the one depicted in FIGS. 10A to 10C. In the simplest embodiments, the subject delivery means is a hollow tube or sheath (or sleeve) of biocompatible material, as described above (e.g., Teflon), which fits around the graft vessel and can be readily removed from the graft vessel following attachment of both ends of the graft vessel to the side of the host vessel(s). The inner diameter of the sheath may vary greatly depending on the graft vessel, but is generally sufficient to allow the vessel to move in the sheath, i.e. for the sheath to be slidably positioned around the prepared graft vessel such that the sheath can be longitudinally slid along the length of the vessel. The opening of the sheath or sleeve is preferably angled in a manner that coincides with the joint angle (Θ in FIGS. 1) of the first structural means.

The opening 61 of delivery means 60 has a cross-sectional area that is smaller than the cross-sectional area of the unconstrained lip of the first structural means on the prepared graft. As such, the lip of the prepared graft vessel must be folded in order for the prepared graft vessel to be positioned within the delivery means, as described above.

In certain embodiments (not shown), the end of the delivery means having opening 61 will be narrowed such that it can pass through an opening in the side of the host vessel that is smaller that the cross-sectional area of the body of the delivery means.

The delivery means may further comprise a retractable tip that narrows the distal end 61 of the delivery means such that the distal end can be readily inserted into an opening in the side of the host vessel. This retractable tip is generally fabricated from a flexible biocompatible material, such as those described above, where the biocompatible material may be polymeric, metallic, etc.

The delivery means may also include a cutting means for making the opening in the host vessel, as described above (in certain embodiments, the retractable tip serves as a cutting means). In addition, the delivery means may include a crimped or highly flexible region, which provides flexibility of the delivery means.

The delivery means may further include a ridge or other suitable means on the inside of the delivery means which provides a reference to determine the location of the first and second structural means of the prepared graft vessel when located within the delivery means. This ridge or other similar means stabilizes the anastomosis system with the graft and structural means within the delivery system, allowing the surgeon to manipulate the prepared graft vessel without touching it. In addition, the ridge or other suitable reference means allows the surgeon to know the position of the second structural means inside the delivery means, which in turn allows the surgeon to know how far to pull back the delivery means in order to exposed the second structural means.

In certain embodiments, the delivery means may also include a means for impeding rotational movement of the prepared graft vessel within the delivery means, such that the orientation of the graft vessel within the delivery means remains substantially constant during attachment of the graft vessel to the host vessel. This means for impeding or preventing rotational movement of the graft within the delivery means may be any convenient structure that provides this function. Suitable structures include mating ridges or protrusions on the inner surface of the delivery means and the outer surface of the first structural means, where these mating structures interact in such a manner as to allow longitudinal movement of the graft within the delivery means but not rotational movement of the graft within the delivery means.

Turning now to the sheath represented in FIGS. 10A to 10C, the delivery means depicted in these figures includes a sheath, a retractable tip (which in certain embodiments also serves as an incision or cutting means), a crimped region and a perforated stripe. FIG. 10B provides a side view of the device where the tip is retracted, while FIG. 10C shows the same device with the cutting means exposed. In FIG. 10B, delivery means 60 includes sheath 62 which has a crimped region or domain 64 and a longitudinal perforation 66, where the longitudinal perforation 66 provides for ready removal of the delivery means following attachment of the graft vessel to the host. Delivery means 60 further comprises retractable tip 65 under the control of actuator 67. FIG. 10A provides an end-on view of the delivery means with the tip retracted.

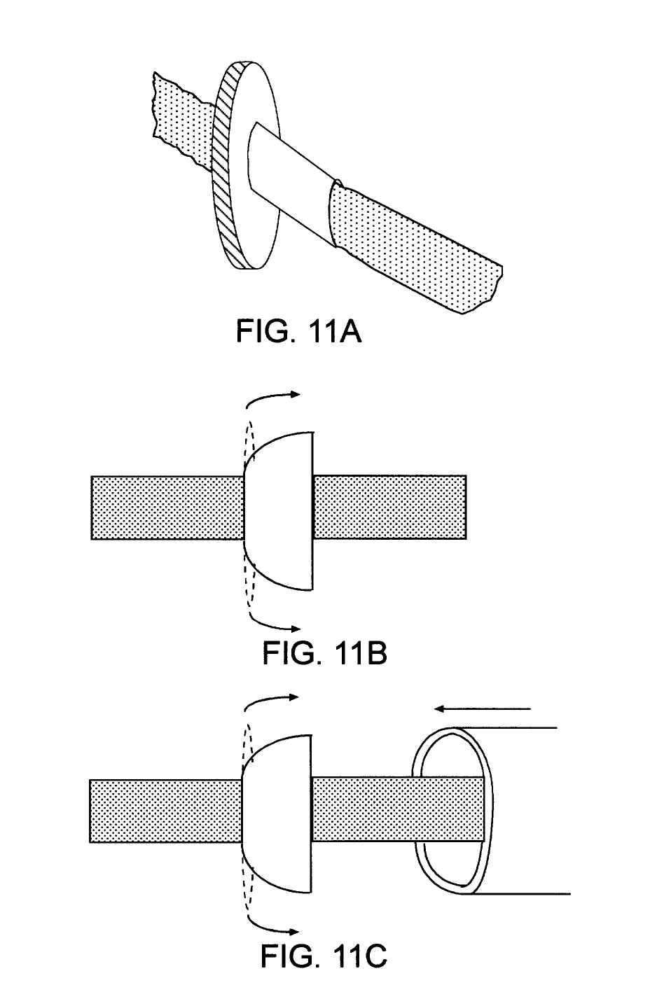

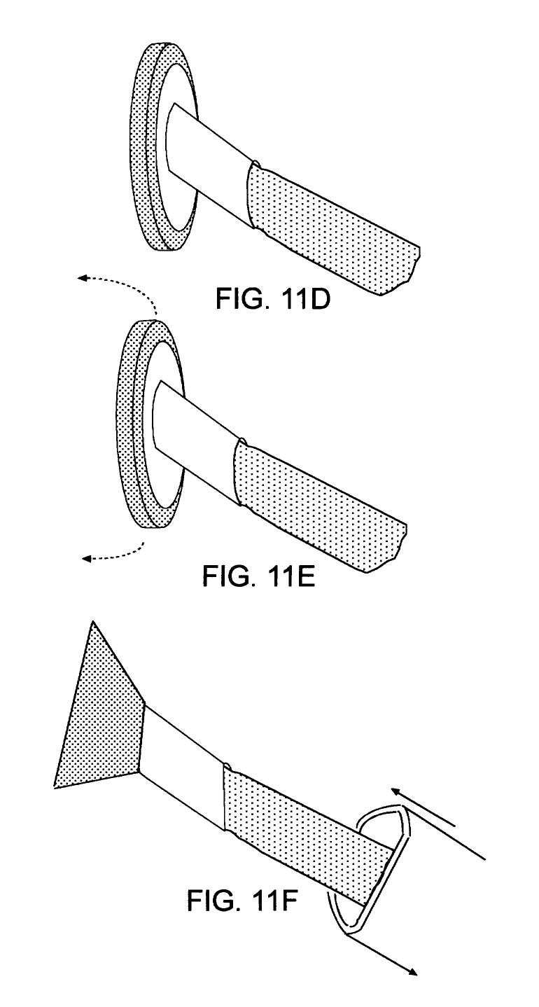

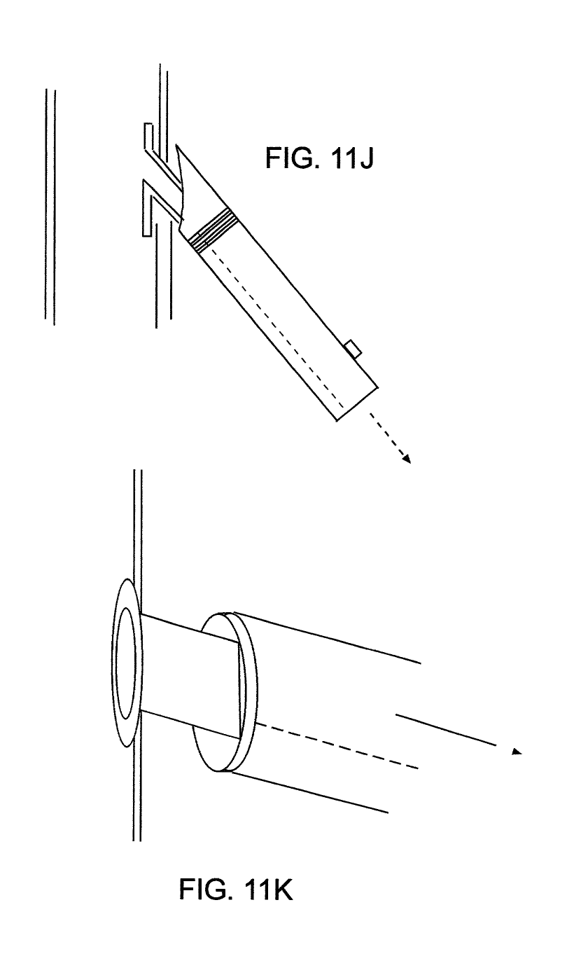

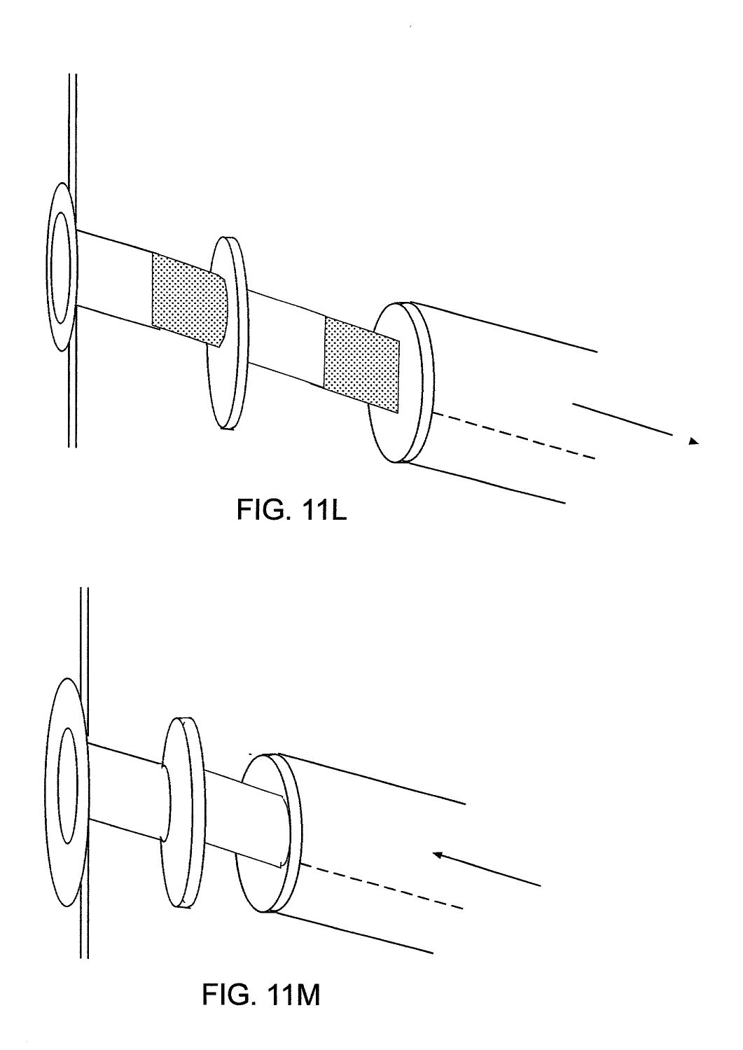

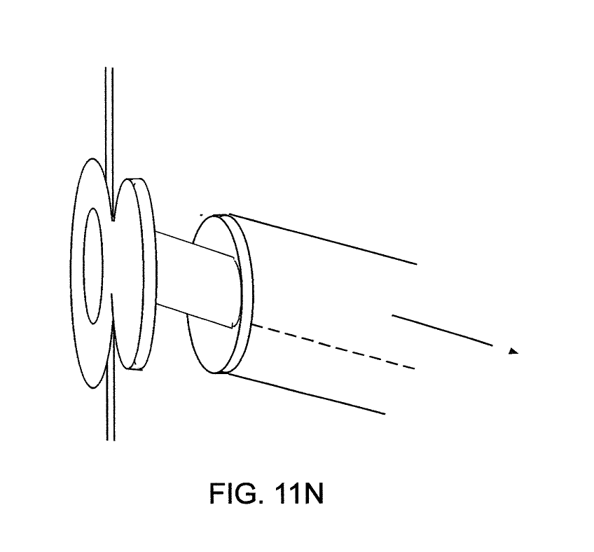

The delivery means depicted in FIGS. 10A to 10C can be employed to stably attach a prepared graft vessel as depicted in FIG. 5 to the side of a host vessel to achieve an anastomosis as shown in FIG. 6, using the protocol depicted in FIGS. 11A to 11N. FIG. 11A, shows the second structural means on a prepared graft vessel. In the first step, the lip of the second structural means is folded back as shown in FIG. 11B. The sheath of the delivery means is then moved over the folded back lip, as shown in FIG. 11C. Next the lip of the first structural means on the prepared graft vessel (as shown in FIG. 11D) is folded forward as shown in FIG. 11E. The sheath of the delivery means is then moved over the folded lip of the first structural means as shown in FIG. 11F to produce the structure shown in FIG. 11G, where the folded back lip of the second structural means is completely within the delivery sheath and the lip of the first structural means is completely folded forward. Next, the proximal end of the delivery means is enclosed by the retractable tip and introduced into an opening that is made in the side of the host vessel. Once positioned in the side of the host vessel, the actuator is moved in the direction shown in FIG. 11H to retract the tip. Upon removal of the tip, the folded up lip of the first structural means which is now inside the host vessel expands to its unconstrained state, as shown in FIG. 11I. The whole system, i.e., graft vessel, anastomosis system and delivery means, is then moved gently away from the host vessel as shown in FIG. 11J until resistance is felt, which indicates that the lip is next to the intimal surface of the host vessel. Next, the sheath of the delivery means is further retracted as shown in FIG. 11K until the second structural means is fully exposed. See FIG. 11L. Upon exposure, the lip of the second structural means assumes its unconstrained configuration, as shown in FIG. 11M. The sheath of the delivery means is then moved towards the host vessel as shown in FIG. 11N which pushes the second structural means onto the first structural means and adjacent to the host vessel wall, thereby stably securing the graft vessel to the host vessel, as shown in FIG. 6.

Utility

The subject methods find use in a variety of different applications in which the joining of a first or graft vessel to a second or host vessel is desired. In other words, the subject methods and devices can be used to join any two vessels together such that fluid and/or gaseous communication between the lumens of the two joined vessels is established, e.g., the graft and host vessels are joined into fluid communication. More specifically, the methods and devices can be used to join the end of a graft vessel to the side of a host vessel, such that fluid or gas, usually fluid, is capable of flowing from the graft vessel into the host vessel. As such, the subject methods and devices are suited for performing any type of the end-to-side anastomosis.

A variety of different vessels can be joined to each other with the subject devices and methods. Typically, the vessels are naturally occurring biological vessels or synthetic mimetics thereof. The vessels may vary greatly in diameter depending on the specific nature of the vessel. Naturally occurring or physiological vessels that may be joined according to the subject invention include airways (e.g., bronchial tubes, etc.), blood vessels (e.g., arteries, veins, etc.), bowel segments (e.g., intestinal vessels, such as small intestine) and urogenital tubes (e.g., ureters, ducts, fallopian tubes, etc) and the like.

The subject methods may also be employed to join a synthetic vessel or tubule to a host vessel. A variety of different types of synthetic vessels have been developed for use in various medical procedures. Synthetic vessels of interest include those described in U.S. Pat. Nos. 5,849,036; 5,607,478; 5,556,426; 5,360,443 and 4,601,718; the disclosures of which are herein incorporated by reference.

Of particular interest in many embodiments is the use of the subject invention to join vascular vessels together in an end-to-side manner, i.e., to perform a vascular end-to-side anastomosis. In vascular end-to-side anastomoses performed according to the subject invention, the end of a graft vascular vessel is typically joined to the side of a host vascular vessel. The graft vascular vessel may be a variety of different types of vessels, where graft vascular vessels typically employed in vascular end-to-side anastomosis procedures include: saphenous vein grafts, LIMA grafts, and the like, as well as synthetic grafts. The host vessel may be an artery or a vein, but is typically an artery, e.g., a coronary artery, the aorta, and the like. Specific vascular end-to-side anastomosis applications in which the subject invention finds use include: coronary artery bypass graft (CABG) procedures, vascular grafting procedures, and the like. Where the subject methods are employed to perform coronary artery bypass grafts, the subject procedures may be open chested or close chested, and may or may not be performed with the heart beating. The subject invention is also suited for use with various minimally invasive port access protocols, in which all of the manipulations are performed using remote access means through small ports made into the chest cavity. The various components of the subject anastomosis system may be readily adapted for use in port access and analogous minimally invasive procedures. The subject systems are particularly suited for use in proximal anastomosis applications, in which the graft vessel is anastomosed to the side of the aorta using the subject systems and methods.

The subject devices and methods may be used with a variety of different hosts in need of an end-to-side anastomosis. Typically the host is a mammalian host, where hosts of interest include domesticated animals, such as livestock, e.g. horses; pets, e.g., dogs, cats; rare or valuable animals, e.g., zoo animals; and humans. In many embodiments, the host will be a human.

Kits

Also provided are kits for use in performing an end-to-side anastomosis according to the subject invention. The subject kits at least include an anastomosis system according to the subject invention, where the anastomosis system is made up of the first and second structural means which may be separate or partially nested, so as to be presented in the kit as unified device. In certain embodiments, the kits will include a plurality of sets of structural means of various sizes, where the number of different structural means in the kits of these embodiments is at least 2, may be at least 4 and may be a high 5, 10 or 20 or higher, depending on the particular application for which the kit is manufactured. In other embodiments, the kit will include a single system of a first and second structural means. The kits may further include a delivery means for use in joining the vessels with the structural means, as described above. In certain embodiments, the kits may further include a synthetic vessel, where the synthetic vessel may or may not be pre-prepared with the structural means, i.e. threaded through the structural means as shown in FIG. 5, and/or pre-loaded into a delivery means. The kits may further include a separate cutting means for use in making the opening in the side of the host vessel. The kits may also include a structural means holding tool for use in graft preparation, particularly graft end eversion. This holding tool may be a stand or analogous holding means that is structured or configured to hold the structural means during graft eversion and attachment to the first structural means, e.g., a holder that fits over and effectively lengthens the elongate portion of the first structural means, where the holder can be removed following eversion. In addition, the kits will also generally include instructions for using the structural means and other contents of the kit to perform an end-to-side anastomosis, where the instructions are present on one or more of: the packaging, the components of the kit (e.g. the structural means), or a package insert.

The following examples are offered by way of illustration and not by way of limitation.

### EXPERIMENTAL

Coronary Artery Bypass

The following is a protocol for performing a coronary artery bypass using the methods and devices of the subject invention to join a graft saphenous vein proximally to the aorta and distally to a coronary artery.

1. The patient is prepared for surgery according to standard CABG procedure.

2. The saphenous vein graft is harvested and excess tissue is removed from the graft vessel, i.e., the graft vessel is cleaned.

3. The first and second structural means as shown in FIG. 7 and 8 are then slid over the graft (i.e., the graft is then threaded through a first and second structural mean, as shown in FIGS. 3A and 3B).

4. The end of the graft is then everted and secured to the lip of the first structural means with the spikes on the upper surface of the lip.

5. Using standard minimally invasive procedures without stopping the heart from beating, an opening is made in the side of the aorta. The lip of the first structural means with the everted end of the graft vessel is then slid into the opening using a motion much like that used when buttoning a shirt, where the button is slid through the button hole. Next, the second structural means is moved onto the first structural means to assume a nested configuration, where the lower surface of the lip of the second structural means presses down on the upper surface of the aorta. This pressure secures the graft to the host vessel and completes the proximal anastomosis. This step 5 can be performed in 60 seconds.

It is evident from the above results and discussion that improved methods for performing end-to-side anastomoses, and devices for use therein, are provided. The subject invention provides a simple and rapid way to perform a wide variety of anastomoses, including vascular anastomoses, such as the proximal anastomosis performed in coronary artery bypass graft procedures. In general, vessels can be joined according to the subject methods without complicated maneuvers and in a short period of time, e.g., less than 60 seconds. Furthermore, exceptional strength is achieved at the site of attachment by the effect produced through nesting of the first and second structural means. The methods and devices can be used in procedures in which the heart is beating. As such, the subject invention is suitable for use in procedures where the patient is not under cardiopulmonary bypass and the patient's heart has not been arrested. Another important advantage of the subject invention is that it establishes intima-to-intima contact between the graft and host vessels. In addition, the structural means provide support for the graft vessel, thereby preventing collapse of the graft vessel which can occur when other anastomosis procedures are employed. The devices employed in the subject methods are simple and can be produced at low cost. In sum, for the reasons provided above (and others), the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. An anastomosis system comprising:
first and second structural means, wherein each of said first and second structural means comprises a tubular region that terminates in a lip at one end, and further wherein said first and second structural means are capable of assuming a nested configuration.

2. The anastomosis system according to claim 1, wherein the inner diameter of the tubular region of said second structural means is greater than the outer diameter of the tubular region of said first structural means.

3. The anastomosis system according to claim 1, wherein said first and second structural means are flexible.

4. The anastomosis system according to claim 3, wherein said first and second structural means are rigid.

5. The anastomosis system according to claim 1, wherein said first and second structural means are nested.

6. The anastomosis system according to claim 1, wherein said first and second structural means are separate.

7. An anastomosis system comprising:
first and second structural means, wherein each of said first and second structural means comprises a rigid biocompatible material and has a tubular region that terminates in a lip at one end, and further wherein said first and second structural means are capable of assuming a nested configuration.

8. The anastomosis system according to claim 7, wherein the inner diameter of the tubular region of said second structural means is greater than the outer diameter of the tubular region of said first structural means.

9. The anastomosis system according to claim 7, wherein said first and second structural means are nested.

10. The anastomosis system according to claim 7, wherein said first and second structural means are separate.

11. The anastomosis system according to claim 7, wherein each lip of said first and second structural means has a curvilinear configuration.

12. The anastomosis system according to claim 11, wherein said curvilinear configuration of said lip of said first structural means is different from said curvilinear configuration of said lip of said second structural means.

13. The anastomosis system according to claim 7, wherein the length of the tubular region of the second structural means is less than the length of the tubular structure of the first structural means.

14. A vessel inserted through first and second structural means, wherein said first and second structural means each have a tubular region that terminates at one end in a lip, and wherein said first and second structural means are capable of assuming a nested configuration.

15. The vessel according to claim 14, wherein the end of said vessel is everted over the distal surface and at least a portion of the proximal surface of the lip of said first structural means.

16. The vessel according to claim 14, wherein the inner diameter of the tubular region of said second structural means is greater than the outer diameter of said tubular region of said first structural means.

17. The vessel according to claim 14, wherein said vessel is a naturally occurring vessel.

18. The vessel according to claim 17, wherein said vessel is a vascular vessel.

19. The vessel according to claim 14, wherein said vessel is a synthetic vessel.

20. The vessel according to claim 14, wherein said first and second structural means are flexible.

21. The vessel according to claim 14, wherein said first and second structural means are rigid.

22. A method for performing an end-to-side anastomosis, said method comprising:
(a) preparing an opening in a host vessel;
(b) inserting into said opening the end of a prepared graft vessel having a graft vessel threaded through first and second structural means, wherein each of said first and second structural means has a tubular region that terminates at one end in a lip, wherein the end of said graft vessel is everted over the distal surface and at least a portion of the proximal surface of the lip of the first structural means; and
(c) sliding said second structural means over the tubular region of said first structural means in a manner sufficient to stably attach said graft vessel to said host vessel and establish fluid communication between the lumens of said graft and host vessels;
whereby said end-to-side anastomosis is performed.

23. The method according to claim 22, wherein said method further comprises preparing said graft vessel.

24. The method according to claim 23, wherein said graft vessel is prepared by:
sequentially inserting said graft vessel through the tubular regions of said first and second structural means; and
everting the end of said graft vessel over the distal surface and at least a portion of the proximal surface of the lip of said first structural means.

25. A kit for use in performing an end-to-side anastomosis, said kit comprising:
an anastomosis system comprising at least one set of first and second structural means, wherein said first and second structural means each have a tubular region that terminates in a lip at one end, and further wherein said first and second structural means are capable of assuming a nested configuration.

26. The kit according to claim 25, wherein the inner diameter of said tubular region of said second structural means is greater than the outer diameter of said tubular region of said first structural means.

27. The kit according to claim 25, wherein said kit comprises a plurality of said anastomosis systems.

28. The kit according to claim 25, wherein said kit further comprises a synthetic vessel.

29. The kit according to claim 25, wherein said kit further comprises a delivery means.

30. The kit according to claim 25, wherein said kit further comprises a structural means holder for use in preparation of a graft vessel.
